(19) United States
(12) Patent Application Publication
(10) Pub. No.: US 2008/0031817 A1
(43) Pub. Date: Feb. 7, 2008

(54) Inhibition Of Superoxide Dismutase By Tetrathiomolybdate: Identification Of New Anti-Angiogenic And Antitumor Agents

(76) Inventors: Andrew P. Mazar, San Diego, CA (US); Steven Pirie-Shepherd, Cardiff, CA (US); Oscar Betancourt, San Diego, CA (US)

Correspondence Address: MCKENNA LONG & ALDRIDGE LLP, 1900 K STREET, NW, WASHINGTON, DC, 20006, US

(21) Appl. No.: 10/590,483

(22) PCT Filed: Feb. 24, 2005
(86) PCT No.: PCT/US05/05767

§ 371 Date: Aug. 20, 2007

Related U.S. Application Data
(60) Provisional application No. 60/546,945, filed on Feb. 24, 2004.

(51) Int. Cl. A61K 31/426; A61K 31/428; A61K 31/554; A61P 35/00; C12N 9/99; C12Q 1/26

(52) U.S. Cl. 424/9.2; 435/184; 435/25; 514/211.06; 514/367; 514/370

(57) ABSTRACT

Though copper is elevated in the tumor tissue and plasma of patients with various malignancies, the molecular targets for copper binding agents in angiogenesis and tumor progression remain poorly understood. It is disclosed that one anti-angiogenic target for the copper binding agent tetrathiomolybdate is intracellular CuZn-superoxide dismutase (SOD1). A second generation tetrathiomolybdate analog, ATN-224, inhibits endothelial cell (EC) proliferation in vitro, binds to SOD1 and inhibits its activity without displacing bound copper ATN-224 can accumulate in ECs and inhibit CuZnSOD activity with an IC₅₀ similar to the IC₅₀ for EC proliferation, resulting in increased generation of intracellular reactive oxygen species. Inhibition of EC proliferation by ATN-224 in vitro is substantially reversed by a synthetic porphyrin SOD mimetic. Similar results were observed in vivo, where inhibition of angiogenesis by ATN-224 in a Matrigel plug model was also reversed by MnTBAP. Thus, a distinct molecular target for copper depletion therapy has been identified and SOD1 is now validated as a target for anti-angiogenesis. Methods for screening, or designing, such SOD1 inhibitors for use as angiogenesis inhibitors and anti-cancer agents are disclosed.

### BACKGROUND OF THE INVENTION

1. Field of the Invention

The present invention in the field of biochemistry and medicine is directed to novel methods for inhibiting angiogenesis and treating tumors and cancer using a tetrathiomolybdate (TM) compound.

2. Description of the Background Art

Angiogenesis, the formation of new capillaries form pre-existing ones (Folkman, J., N. Engl. J. Med., 1971, 285:1182-1186; Hanahan D. et al., Cell, 1996, 86:353-364), is a normal part of embryonic development, wound healing and female reproductive function. However, angiogenesis also plays a pathogenic role in the establishment and progression of certain diseases. Cancer, rheumatoid arthritis and diabetic retinopathy are examples of such diseases (Carmeliet P. et al., Nature, 2000, 407:249-257). Angiogenesis is of profound importance in the transition of tumors from a dormant state to a malignant state (Folkman, J, 1990, JNCI 82: 4-6). Anti-angiogenic therapy holds promise in inhibiting the progression of these diseases.

Angiogenesis can be triggered by several pro-angiogenic cytokines. In the setting of cancer, tumor cells under hypoxic conditions secrete vascular endothelial growth factor (VEGF) and/or fibroblast growth factor (bFGF). These proteins diffuse and bind to specific receptors on endothelial cells (ECs) in the local vasculature, perturbing the balance of pro- and anti-angiogenic forces in favor of angiogenesis. As a consequence of binding these proteins, ECs are activated to (a) secrete enzymes that induce remodeling of the associated tissue matrix, and (b) change the patterns and levels of expression of adhesion molecules such as integrins. Following matrix degradation, ECs proliferate and migrate toward the hypoxic tumor, resulting in the generation and maturation of new blood vessels.

The concept has emerged that, due to the abundance of pro-angiogenic factors, these anti-angiogenic molecules are unable to overcome the pro-angiogenic balance in a primary tumor. However, since they are secreted into circulation, these anti-angiogenic molecules are capable of inhibiting angiogenesis at other locations where tumor cells may have begun to invade. Consequently, micro-metastases comprising these tumor cells at these new locations remain dormant. This hypothesis explains a puzzling observation made by surgeons many years ago: at various times after surgical removal of a primary tumor in a patient with no obvious metastatic disease, the patient returns with advanced metastatic disease. Thus, clinical intervention by treatment with one or more anti-angiogenic factor could inhibit the angiogenic process and halt tumor growth as well as metastasis. Significant evidence in the literature (cited above) supports this notion.

### Role of Copper in Biology and Disease

Copper plays an essential role in a number of biochemical and biological processes including mitochondrial function, cellular iron metabolism, cross-linking of connective tissue, vascular response to injury and detoxification of free radicals. Copper is a co-factor for a number of enzymes including CuZn-superoxide dismutase (SOD1) and cytochrome C oxidase. Enzymatic activity of SOD1 is absolutely dependent on copper. Other metal chelators inhibit SOD1 and also have an impact on tumorigenesis and angiogenesis. Tetraethylthiuram disulfide (Disulfiram; “DS”) and diethyldithiocarbamate (DTC) both inhibit SOD activity in vivo (Forman H J et al., 1980, J. Pharmacol. Exp. Ther. 212:452-5. Only DTC inhibited SOD in vitro. DS is catabolized to DTC in vivo, resulting in the in vivo effect of DS. However, these other metal chelators are not specific for copper and could also inhibit SOD1 activity by binding to the zinc co-factor required for SOD1 activity. Tetrathiomolybdates (TMs) are absolutely specific for copper and do not bind to other divalent metal cations.

Ohman, L et al., 1986, Clin Sci (Lond). 70:365-9 disclosed that DS administered to alcoholics reduced by 20% extracellular SOD activity compared to untreated alcoholics and healthy control. DS did not inhibit SOD activity in vitro.

Jornvall et al., 1987, Enzyme 37:5-18, reviewed studies of aldehyde dehydrogenase and alcohol dehydrogenase, target enzymes for DS. These enzymes are tetramers wherein the subunits do not form intrachain type bonds and no functional metal is present. DS interacted with Cys 302, which is important for enzyme activity and appears to be the basis for DS efficacy in alcoholics.

Pyatt et al., 2000, BBRC 274:513-518, disclosed that both diethyl and dimethyl DTCs inhibited (i) NFκB activity and (ii) hematopoiesis by a copper-dependent mechanism that was not mediated by NFκB. DTC increased intracellular copper levels which potentiated DTC toxicity.

Iseki , A et al., 2000, BBRC 276 88-92, studying human aortic smooth muscle cells, disclosed that pyrrolidine DTC (PDTC) inhibited TNFα-dependent activation of NFκB by increasing intracellular copper levels. This increase could be abrogated by bathocuproinedisulfonic acid (BCS), a cell-impermeable chelator of Cu⁺.

From these observations, the present inventors inferred that DTCs reduce Cu⁺⁺ to Cu⁺, which is the copper state required for binding the cellular copper transporter, CTR1. Thus only the Cu⁺ form can enter a cell via CTR1.

Lipsky et al., 2001, Chem, Biol. Interact 130/132:81-91, disclosed that DS inhibits aldehyde dehydrogenase irreversibly by inducing the formation of an intramolecular disulfide bond at Cys302, which is a functionally important residue for enzymatic activity. DTC also induces formation of a disulfide bond at this Cys.

An appreciation for the multifarious role of copper ions in cancer have come from several directions. It has long been known that serum copper levels are elevated in (a) experimentally induced malignancies (Chakravarty, P K et al., 1984, J Comp Pathol. 94:603-6 and (b) patients with a variety of tumors (Chakravarty, P K et al., 1984, J Cancer Res Clin Oncol. 108:312-15). Mashiba, H et al., 1990, Jpn J Exp Med. 60:209-14, used DS as an inactivator of SOD in combination with ascorbic acid (AsA) as an inhibitor of catalase to examine the roles of oxygen free radicals in the inhibition of tumor cell proliferation. Simultaneous addition of DS and AsA to two tumor cell lines inhibited cell proliferation. Pretreatment of the cells with the combination potentiated the inhibition. This inhibitory effect was prevented by adding catalase. The authors concluded that DS+AsA increases of intracellular oxygen free radicals in tumor cells.

A number of growth factors and cytokines with either pro- or anti-neoplastic function, such as VEGF, bFGF, TNFα and IL-1 are copper-dependent. Intracellular copper regulates the stress-induced release of the cytokines IL-1α and FGF-1. The main copper-binding protein in the body, ceruloplasmin (Cp) is elevated in the serum in advanced stages of solid malignant tumors (Senra-Varela A, 1997, Cancer Lett. 121:139-45) and melanoma (Ros-Bullón et al, 2001, Anticanc. Res. 21:629-32.

Patent publications of Marikovsky (Int'l Pat. Publ. WO 99/34763 and WO 99/34784 and its corresponding U.S. Pat. No. 6,288,110) disclosed, respectively that Thiram (tetramethyl thiuram disulfide; “TMTD”) and DS inhibited angiogenesis and inflammation, and would be useful in treating angiogenesis-dependent pathologies, including neoplasms, and in preventing cell hyperproliferation and formation of clots along or around medical devices. These documents do not mention inhibition of SOD. Marikovsky also disclosed that TMTD directly inhibited SOD activity in vitro and angiogenesis in vivo. (Brit. J. Cancer 86 779-787, 2002) Note that this result differs from DS, which does not inhibit SOD in vitro.

U.S. Pat. No. 6,548,540 (to Kennedy) discloses DTC derivatives as anti-cancer agents. U.S. Pat. No. 6,589,987 (to Kennedy) discloses DS as an anti-cancer agent and recommends that DS be administered with a heavy metal to enhance its anticancer effect. Neither of these documents discloses SOD inhibition.

Furuta, S et al., 2002, Biochem. J. 365639-648 tested the ability of PDTC, DTC and ethylene(bis)-DTC to oxidize the oncogenic protein p53 in cultured human breast cancer cells. All three compounds increased copper levels. Although copper accumulation at lower levels (25-40 μg/g of cellular protein) increased the steady-state levels of p53, only at levels >60 μg/g of cellular protein, induced only by PDTC, was p53 was oxidized. BCS inhibited this oxidation. These results suggest that p53 oxidation depends on intracellular copper concentration and is vulnerable to free radical-mediated oxidation at Cys residues.

The tetrathiomolybdate (TM) dianion has a very specific and high affinity for copper ions (Ka˜10⁸ M⁻¹), and forms tripartite complexes with copper and protein. Animal studies demonstrated that TM removed copper from proteins such as metallothioneins (Suzuki, K T et al., 1993, Toxicology 83:149-158) and repartition it to albumin for excretion (Suzuki, K T et al., 1995, J. Trace Elements Med. Biol. 9:170-175). These tripartite complexes appear to involve the formation of copper-molybdenum-sulfur clusters, with some of the coordinating sulfurs being supplied by the side chains of appropriate amino acids in target proteins (George G N et al., 2003, J Am Chem Soc 125:1704-5).

In vitro, TM decreased the production of several pro-angiogenic mediators in an inflammatory breast carcinoma tumor xenograft model (Pan Q et al., 2002, Cancer Res. 62:4854-59). TM may enhance the efficacy of doxorubicin against breast carcinoma (Pan Q et al., 2003,) Mol Cancer Ther. 2:617-22). TM was antiangiogenic in a murine model of head and neck squamous cell carcinoma (Cox C et al., 2001, Laryngoscope 111:696-701) and was more effective in combination with radiation therapy than alone in another animal model (Khan M K et al., 2002, Neoplasia 4:164-70). TM inhibited tumor growth in the Dunning prostate cancer model (Van Golen, K L et al., 2002, Neoplasia 4:373-3795). TM was tested as an anti-copper therapy in a physician-sponsored phase I trial involving 18 patients with metastatic disease (Brewer G J et al., 2000, Clin Cancer Res. 6:1-10); at the highest dose tested, total body copper fell to a targeted level (measured as serum Cp) in six patients while briefly stabilizing disease in five of them. The possibility of using TM in combination with other therapies was discussed in a recent publication describing a phase II TM trial for advanced kidney cancer (Redman B G et al., 2003, Clin Cancer Res. 9:1666-72).

### SUMMARY OF THE INVENTION

Ammonium tetrathiomolybdate (TM), has been tested in multiple physician-sponsored trials for the treatment of cancer and has shown promising early results. However, this compound has poor stability. A series of more stable tetrathiomolybdate salts have been prepared and characterized (Ternansky et al., WO 04/009034; Merajver et al. WO 04/009072). One of these derivatives, choline tetrathiomolybdate, was developed by the present inventors and their colleagues, and designated ATN-224 (supra). ATN-224 is a small orally available compound that specifically binds copper ions with a high affinity (K_{d} of 10 nM) as determined by calorimetry (ITC) studies. Using standard binding assays, ATN-224 does not bind detectably to other metal cations including Zn²⁺, Fe²⁺, Ca²⁺, Mn²⁺ and Mg²⁺

The present inventors have discovered that ATN-224 inhibits proliferation and angiogenesis independent of its effect on systemic copper levels. ATN-224 accumulates in cells and removes copper from SOD1. ATN-224 inhibits intracellular SOD1 in a dose- and time-dependent manner. ATN-224 treatment causes an accumulation of superoxide anions in cells The effects of ATN-224 can be reversed by SOD mimetics

Tumors treated with ATN-224 have reduced SOD1 enzymatic activity in the face of unchanged levels of the SOD1 protein (measured by Western blot).

Measurement of SOD activity in combination with assays of cell proliferation assay serve as an effective screening method for novel antiangiogenic compounds.

Based on the studies of Brewer and colleagues, it was hypothesized that TM (or in the present case, ATN-224) mediated its effect by depleting copper systemically. This effect eventually ‘trickled down’ to the cell. However, the present inventors have now obtained direct evidence of a different mode of action.

According to the present invention, ATN-224 provided to cells or administered to a subject, it is taken up by cells and inhibits SOD1 by removing copper from the enzyme. SOD1 inhibition is directly linked to inhibition of cell proliferation. ATN-224-mediated inhibition of cell proliferation and angiogenesis is reversed by SOD mimetics in a proliferation assay or a Matrigel angiogenesis model in vivo, respectively. In ECs treated with ATN-224 superoxide anions accumulate, so that the inhibition of SOD1 can lead to oxidative damage of cellular proteins involved in signaling and survival. The fact that ATN-224 down-regulates IL-8 expression is an indication that NFκB dependent signaling may be involved.

Thus, according to this invention, ATN-224 therapy reduces SOD1 activity in tumor cells. The present invention differs from the approaches of Marikovsky and of Kennedy (supra) in the following ways.
(1) ATN-224 is antiangiogenic and anti-tumorigenic in vitro and in vivo.
(2) ATN-224 is highly very specific for copper and does not bind other metals (Ca²⁺, Fe²⁺ Mg²⁺, Zn²⁺, Mn²⁺)
(3) ATN-224 inhibit SOD1 in vitro but does not similarly inhibit enzymes that do not utilize copper as a co-factor. Thus ATN-224 is more specific than dithiocarbamates; and does not inhibit, for example, aldehyde dehydrogenase.
(4) ATN-224 accumulates in cells and inhibits intracellular SOD1, inducing a measurable increase in intracellular superoxide concentration. This mode of action is specific. As a consequence, ATN-224 will lead to reduction in the concentration of NO ions, because superoxide anions react with NO ions to form peroxynitrite. NO is required for EC homeostasis. Peroxynitrite causes tyrosine nitration of intracellular proteins, which is cytotoxic.
(5) ATN-224 does not increase intracellular copper levels, i.e., is not a general antioxidant. Rather, it has very specific and defined effects on key enzymes in the cell.

ATN-224 inhibits EC proliferation, and the addition of copper to ATN-224 abrogates this inhibition.

ATN-224 is expected to inhibit other enzymes for which copper is an essential co-factor. These include, but are not limited to, extracellular SOD, lysyl oxidase, and cytochrome C oxidase. ATN 224 may have greater or lesser effects on these enzymes.

ATN-224 will inhibit the proliferation of any cell type sensitive to increased superoxide concentrations. This includes, but is not limited to, proliferating ECs, tumor cells, and inflammatory cells in contrast to quiescent cells.

The present invention provides novel methods to inhibit or reduce angiogenesis, tumor growth and EC proliferation using ATN-224.

Transition metals and induction of oxidative stress have been implicated in the etiology of non-cancerous diseases, especially, neurodegenerative diseases such as Alzheimer's disease, Parkinson's disease and amyotrophic lateral sclerosis (ALS). Thus, the present invention also provides compositions and methods for the treatment of any disease whose pathobiology involves abnormal presence or undesired action of transition metals, including conditions where the presence of the transition metal may induce oxidative stress.

An anti-angiogenic and anti-tumor pharmaceutical composition comprises an effective amount ATN-224; and a pharmaceutically acceptable carrier.

Preferably the pharmaceutical composition is in a form suitable for oral administration. In another embodiment, the composition is suitable for injection.

Also included is a method for treating a subject having a disease or condition associated with undesired EC proliferation and angiogenesis, comprising administering to the subject an effective amount of the pharmaceutical composition comprising ATN-224. A preferred disease or condition for this treatment is a tumor or cancer.

Thus, in the above methods, the subject has a tumor, and the inhibition of cell proliferation-inhibition and/or angiogenesis results in reduction in size or growth rate of the tumor or destruction of the tumor. Preferably, the subject is a human.

Other diseases or conditions against which the above method is effective include primary growth of a solid tumor, leukemia or lymphoma; tumor invasion, metastasis or growth of tumor metastases; benign hyperplasia; atherosclerosis; myocardial angiogenesis; post-balloon angioplasty vascular restenosis; neointima formation following vascular trauma; vascular graft restenosis; coronary collateral formation; deep venous thrombosis; ischemic limb angiogenesis; telangiectasia; pyogenic granuloma; corneal disease; rubeosis; neovascular glaucoma; diabetic and other retinopathy; retrolental fibroplasia; diabetic neovascularization; macular degeneration; endometriosis; arthritis; fibrosis associated with a chronic inflammatory condition, traumatic spinal cord injury including ischemia, scarring or fibrosis; lung fibrosis, chemotherapy-induced fibrosis; wound healing with scarring and fibrosis; peptic ulcers; a bone fracture; keloids; or a disorder of vasculogenesis, hematopoiesis, ovulation, menstruation, pregnancy or placentation associated with pathogenic cell invasion or with angiogenesis.

A preferred disease or condition to be treated by the above method is tumor growth, invasion or metastasis. This includes brain tumors. Examples of such brain tumors are astrocytoma, anaplastic astrocytoma, glioblastoma, glioblastoma multiformae, pilocytic astrocytoma, pleiomorphic xanthoastrocytoma, subependymal giant cell astrocytoma, fibrillary astrocytoma, gemistocytic astrocytoma, protoplasmic astrocytoma, oligodendroglioma, anaplastic oligodendroglioma, ependymoma, anaplastic ependymoma, myxopapillary ependymoma, subependymoma, mixed oligoastrocytoma and malignant oligoastrocytoma.

The method is also used to treat a uterine disease such as endometriosis and pathogenic ocular neovascularization such as that associated with, or a cause of, proliferative diabetic retinopathy, neovascular age-related macular degeneration, retinopathy of prematurity, sickle cell retinopathy or retinal vein occlusion.

In another embodiment, the present invention provides a method for identifying from among a plurality of existing compounds a molecule that is useful as a copper-binding angiogenesis inhibitor and/or anti-cancer agent,
(a) selecting a compound or a plurality of compounds with the following characteristics:

(i) a molecular mass of preferably less than about 1000 Daltons;

(ii) at least two sulfur atoms separated from one another by 2 or 3 atoms; and

(iii) optionally, one or preferably both of the sulfur atoms have a lone pair of electrons;
(b) testing the compound or plurality of compounds selected in (a) above for their ability to remove copper from the SOD1 enzyme or to inhibit the catalytic activity of SOD1, and selecting one or more compounds with such copper-removing or enzyme-inhibitory activity; and
(c) optionally, testing the compound or compounds selected in (b) for their ability to inhibit cell proliferation, preferably of activated ECs or of tumor cells in vitro.
   wherein a compound that has the characteristics of (a), tests positive for SOD1-inhibitory activity and, optionally, inhibits cell proliferation in vitro, is identified as being useful as the angiogenesis inhibitor and/or anticancer agent.

Preferably, in the above method, the compound selected in selecting process (a) also has the ability to adopt a conformation that places the two sulfur atoms at a distance of between about 2 Å and 5 Å, more preferably between about 3.4 Å and 3.8 Å, even more preferably between 3.5 Å and 3.7 Å.

The above selecting process (a)may be performed computationally.

In the foregoing method, the assay for SOD1 inhibitory activity is preferably a biochemical assay, preferably one that employs a chromogenic water-soluble tetrazolium salt to yield a measurable colored product. A preferred tetrazolium salt is WST-1, described below.

In the above method, the compound or compounds may also be tested for inhibitory activity in an assay of EC migration and/or EC growth and/or tumor growth in vivo, using, for example, a Matrigel® plug assay.

In the above method the compound preferably inhibits inhibition of SOD1, cell migration, cell growth and/or tumor growth by at least about 10%, preferably by at least about 25%, more preferably by at least about 50%, and even more preferably by at least about 70%.

Also provided is a method of designing a copper-binding molecule that removes copper from SOD1, thereby inhibiting SOD1 enzymatic activity, and is therefore useful as an antiangiogenic and/or anti-cancer agent, the method comprising determining atomic constituents and conformational parameters of the molecule being designed such that the molecule has the following physicochemical characteristics:

(i) a molecular mass of preferably less than about 1000 Daltons

(ii) at least 2 sulfur atoms separated from one another by 2 or 3 atoms

(iii) optionally one, or preferably both, sulfur atoms have a lone pairs of electrons;

and has the following biochemical characteristics:

(i) removes copper from SOD1; or

(ii) inhibits the catalytic activity of SOD1, and
and has one or more of the following effects on cells: (i) inhibits proliferation of activated endothelial cells in vitro, (ii) inhibits proliferation of tumor cells in vitro; (iii) inhibits endothelial cell migration in vitro or in vivo; (iv) inhibits tumor cell growth in vivo;
   thereby designing the molecule. Preferably, the molecule being designed has the ability to adopt a conformation that places the two sulfur atoms at a distance of between about 2 Å and about 5 Å, preferably at a distance of between about 3.4 Å and 3.8 Å, more preferably, between 3.5 Å and 3.7 Å.

The invention includes a method for designing and making a copper-binding molecule, preferably an organic molecule, that removes copper from SOD1, and thereby inhibits SOD1 enzymatic activity, rendering the molecule useful as an antiangiogenic and/or anti-cancer agent, which method comprises:
(a) designing the molecule in accordance with the description above;
(b) selecting a synthetic process that will produce the molecule and stabilize its structure as assessed by any conventional analytical or functional method for the determining stability of a molecule, including, for example, HPLC, TLC, NMR spectroscopy and chemical or biological assays for function such as SOD1 inhibition;
(c) employing the synthetic process of (b) to synthesize the molecule,
   thereby making the molecule.

This method preferably further comprises:
(d) testing the molecule produced in step (c) for one or more of the following activities: (i) removing copper from SOD1; (ii) inhibiting catalytic activity of SOD1, (iii) inhibiting proliferation of activated endothelial cells in vitro, (iv) inhibiting proliferation of tumor cells in vitro; (v) inhibiting endothelial cell migration in vitro or in vivo; or (vi) inhibiting tumor cell growth in vivo.

The present invention is also directed to a method for removing copper from the SOD1 enzyme, comprising contacting a sample comprising SOD1 with an effective amount of a compound identified in accordance with a method described above, or designed or made in accordance with a method described above, for a time sufficient for removal of the copper from the enzyme. The compound may be ATN-427, ATN-714, ANT-719 or ATN-722. The removing may be accomplished in vivo.

Also provided is a method for inhibiting the activity of the SOD1 enzyme, comprising contacting a sample comprising SOD1 with an effective amount of a compound identified as an SOD1 inhibitor in accordance with the method described above, or designed or made in accordance with a method described above, for a time sufficient for inhibition of the enzyme. The compound may be ATN-427, ATN-714, ATN-719 or ATN-722. The contacting may be accomplished in vivo.

Also included is a method for inhibiting endothelial cell proliferation comprising providing to endothelial cells (ex vivo or in vivo) an effective amount of a compound identified as a proliferation inhibitor in accordance with a method described above, or designed or made in accordance with a method described herein, for a time sufficient for inhibition of the proliferation. The compound may be ATN-427, ATN-714, ATN-719 or ATN-722.

### BRIEF DESCRIPTION OF THE DRAWINGS

FIG. 1 is a graph showing that ATN-224 inhibits proliferation of ECs. Cells were incubated with ATN-224 as described in Example I. After 72 hours, cell numbers were quantified using the acid phosphatase assay.

FIG. 2 is a graph showing that ECs incubated with ATN-224 accumulate molybdenum.

FIG. 3 is a graph showing that ATN-224 inhibits the activity of SOD1. SOD was assayed using a xanthine/xanthine oxidase superoxide generation reaction coupled to a water soluble tetrazolium salt, WST-1. SOD activity was assayed in increasing concentrations of ATN-224.

FIG. 4 is a graph showing that ATN-224 depletes copper from the SOD1 enzyme. SOD enzyme was incubated in the presence of increasing concentrations of ATN-224 for 30 minutes. The protein was then purified and copper content assayed.

FIGS. 5A and 5B are graphs showing that ATN-224 inhibits intracellular SOD activity in a time- and dose-dependent manner. FIG. 5B shows inhibition of intracellular SOD in the presence of serum.

FIG. 5C is a Western blot showing that, while incubation with ATN-224 inhibits SOD activity, intracellular SOD protein (antigen) is not depleted.

FIGS. 6A-6C are a series of photomicrographs of ECs incubated with ATN-224, resulting in accumulation of superoxide anions. Cells preincubated with ATN-224 for 72 hours were incubated with 5 μM DHE for 45 minutes before being visualized by fluorescence microscopy. Magnifications are 10× in FIGS. 6A and 6B, and 40× in FIG. 6C.

FIGS. 7A and 7B are graphs showing that a SOD mimetic (MnTBAP) abrogates the inhibitory effect of ATN-224 on EC proliferation (FIG. 7A) and angiogenesis in a Matrigel® plug assay (FIG. 7B).

FIG. 8 is a graph showing the inhibitory effects on SOD1 activity of three novel compounds from a chemical library predicted to have such activity based on their structures. Results of a fourth compound, ATN-427 (choline tetrathiotungstate) are also shown. The activity of ATN-224 is shown as a comparison.

FIG. 9 is a graph showing the effects on EC proliferation of three novel compounds from a chemical library (see FIG. 8) in comparison with ATN-224.

### DESCRIPTION OF THE PREFERRED EMBODIMENTS

The present invention is based on the discovery that ATN-224, a derivative of TM, which exhibits anti-angiogenic activity and anti-tumor activity acts at least in part by removing copper from SOD1, thereby inhibiting the activity of this enzyme, and curtailing the cells ability to defend itself against superoxide generation. ATN-224 also inhibits EC proliferation. Pharmaceutical compositions comprising this compounds or other compound identified using this invention are useful in the treatment of cancer and other diseases associated with aberrant or undesired angiogenesis.

The present invention provides an approach to screening compounds and identifying those with copper-binding activity like ATN-224, which are potential angiogenesis inhibitors and anti-cancer agents. Such screening, based on (1) the present discovery that ATN-224 targets copper specifically, and thereby targets the SOD1 enzyme, and (2) the known chemical structure of ATN-224. Thus a preferred, though not requisite, first step is to identify (or design) a candidate compound that shares certain structural features with ATN-224. The candidate compound is tested for its ability to (a) remove copper from SOD1 and/or inhibit the activity of SOD1. Optionally, the compound is tested in a proliferation assay, for example, of ECs or tumor cells. Based on the inventors' knowledge that ATN-224 targets copper specifically, and thus targets SOD1 activity, it is possible to use the known chemical structure of ATN-224 to identify or design other compounds that share common structural features, and use the SOD assay in conjunction with the proliferation assay to screen such candidate compounds for biochemical and biological activity.

The sulfur-sulfur distance in TM, the active agent of ATN-224, is 3.66 Å as determined geometrically from Cu—Mo, Cu—S, and Mo—S distances from Extended X-ray Absorption Fine Structure (EXAFS) (George et al., supra). Also, polythiaethers are copper-selective and have an S-S distance of 3.48 Å (Kulatilleke, C P et al., 1999, Inorg. Chem. 38:5906-09). The present inventors have selected the following criteria and have searched commercially available database (“Specs”, ca. 235,000 structures) for small molecules that satisfy these criteria:
1. At least 2 sulfur atoms separated by 2-3 atoms.
2. Sulfur atoms have lone pairs of electrons, thereby, for example, excluding sulfones and sulfonamides. Thiols and sulfoides may or may not be excluded. Though it is not necessary that one or both sulfurs have a lone pair of electrons, it is preferred; this property was used in the present screen.
3. Molecular mass is <1000 Da.
4. The ability to adopt a conformation that places two sulfur atoms preferably at a distance of between about 2 Å and about 5 Å, preferably between about 3.4 Å and 3.8 Å, more preferably between 3.5 Å and 3.7 Å.
   As disclosed in Example VII, the present inventors searched the above database for small molecules that satisfy these criteria and found several that tested positive for inhibition of SOD1 and of EC proliferation. Other compounds can be identified in the same matter, e.g. from other databases.

### Measurement of SOD Enzymatic Activity

SOD activity may be measure using any conventional (or yet undiscovered) assay. Most commonly, the assay includes a superoxide generating system, a preferred example of which is xanthine/xanthine oxidase. SOD catalyzes the breakdown of superoxide anion (O₂⁻) by dismutation of two superoxide anions into hydrogen peroxide and molecular oxygen.

2O₂⁻+2H⁺→H₂O₂+O₂

Over the past few decades, various SOD assay methods have been developed. It was recently found that certain water-soluble tetrazolium salts such as XTT, WST-1 and WST-8 are suitable for the detection of O₂⁻ and are applicable to the SOD assay. Of these tetrazolium salts, WST-1 is considered to be the most promising for use in assays due to its sensitivity, low absorbance of the oxidized form and its water solubility (Ukeda, H, Dojindo Newsletter Vol. 3, World Wide Web address “dojindo.com/newsletter/review_vol3-3.html”). The SOD assay method using of WST-1 can be applied to biological/biochemical samples such as erythrocytes, liver and heart tissue.

### Conventional SOD Assays

Because the nature of samples being analyzed may vary widely, assays have focused on high selectivity and minimization of interference from other sample components. For production of O₂⁻, the substrate of SOD, a xanthine-xanthine oxidase reaction is typically employed. A probe for the detection of O₂⁻ is included in the reaction solution. A change in the measured property of the probe in the absence of the test sample is designated as the “blank” control, and the ratio of suppression of the probe's change by the sample is designated the “inhibition ratio.” Commonly, activity is expressed as an IC₅₀ (concentration at which the sample inhibits by 50%). Some O₂⁻ generated by the xanthine-xanthine oxidase reaction is spontaneously transformed to oxygen and hydrogen peroxide. This spontaneous dismutation reaction occurs rapidly in acidic conditions, and at the rate of 8.5×10⁵-8.5×10⁴ M⁻¹ s⁻¹ at physiological pH (pH 7-8). Therefore, the second-order rate constant of the reaction between O₂⁻ and the probe should exceed the rate constant of the dismutation reaction. In the case where the rate constants are almost the same, the concentration of the probe should be increased. Several different types of probes have been used: those which, upon reaction with O₂⁻, change color (colorimetric probes) which are preferred, those that emit light (chemiluminescence probes), or those that produce specific radicals (spin trap agents).

The most common detection method has been spectrophotometric. This method uses either cytochrome C or nitroblue tetrazolium (NBT). The detection of O₂⁻ by the cytochrome C reducing method is based on generation of a purple colored dye from reduced cytochrome C.

Cyt(Fe¹¹¹)+O₂⁻→Cyt(Fe¹¹)+O₂)

This has been is the most common method since the discovery of SOD. This method requires continuous monitoring (e.g., 1.5 minute intervals), so it is not suitable for high-throughput detection. The NBT method is based on the generation of a water-insoluble blue formazan dye (λₘₐₓ=560 nm) by a reaction with O₂⁻. Because its insolubility, the dye creates a non-homogeneous suspension causing problems in reproducibility. The most significant disadvantage of the NBT method is that 100% inhibition cannot be achieved, even with the addition of excessive amount of SOD.

A chemiluminescence probe used for O₂⁻ detection can also be used in an SOD assay. The commonly used probes are lucigenin and a luciferin derivative (MCLA). These reactions are highly pH dependent, so that SOD detection by chemiluminescence under physiological pH conditions is impractical. MCLA also reacts with singlet oxygen. Moreover, MCLA reacts with dissolved oxygen to emit background luminescence, and transitional metal ions accelerate the oxidation reaction.

Electron Spin Resonance (ESR) spectroscopic methods may also be used. At room temperature, the ESR signal of O₂⁻ in solution which cannot be detected directly, is detected indirectly detected by a spin trap method using, for example, 5,5-dimethyl-1-pyrroline N-oxide (DMPO). O₂⁻ trapping DMPO indicates a particular ESR spectrum pattern so that ESR detection is the most specific method for O₂⁻ detection. However, the second-order rate constant between DMPO and O₂⁻ is relatively lower than the reaction constant of the spontaneous reaction of O₂⁻. Therefore, a large amount of DMPO is needed, increasing assay cost. This method also requires a relatively expensive ESR instrument.

More preferred recently developed SOD assays use water-soluble tetrazolium salts, are economical and require a simple instrument, are less pH-sensitive, and are highly O₂⁻-specific. A preferred capability is that the assay determine 100% inhibition of SOD without interference from other components. XTT is a water-soluble tetrazolium salt that has been used as a substrate for the electron transfer system of bacteria cells or mammalian cells. Its structure is indicated below. While NBT has a bis-tetrazolium structure, XTT is monotetrazolium with two sulfonic acid groups. XTT does not appear to interact directly with the reduced form of some enzymes that are generated during the oxidase reaction process. Thus, although XTT has certain advantages over NBT, it is still burdened by pH dependent sensitivity changes. Whereas results with NBT are stable in the range between pH 8 and 10.2, the sensitivity of the assay with XTT decreases as the pH is lowered. Further, water-solubility is less than optimal.

Ishiyama and his group (Ishiyama, M. et al., 1993, Chem. Pharm. Bull., 41:118; Ishiyama et al., Anal. Sci., 12:515 (1996); Ishiyama et al., Talanta, 44:1299 (1997) developed several new water-soluble tetrazolium salts. Since the water-solubility of these compounds range from >10 mM to >100 mM, these compounds were adopted for SOD assay in place of XTT. WST-1 and WST-8 are mono-tetrazolium salts that include have sulfonate group(s).

WST-1 and WST-8 can achieve 100% inhibition at high concentrations of SOD. They have the advantage that very similar IC₅₀ values can be measured in solutions of different pH, thereby overcoming several shortcomings of XTT and NBT. WST-1 appears to permit determination of SOD activity with the highest sensitivity among several other tetrazolium salts and is therefore preferred. Recently, Winterbourn and his group (A V Peskin et al., 2000 Clin. Chim. Acta 293:157) developed a microplate assay for detecting SOD activity with WST-1 and employed it with human erythrocytes and

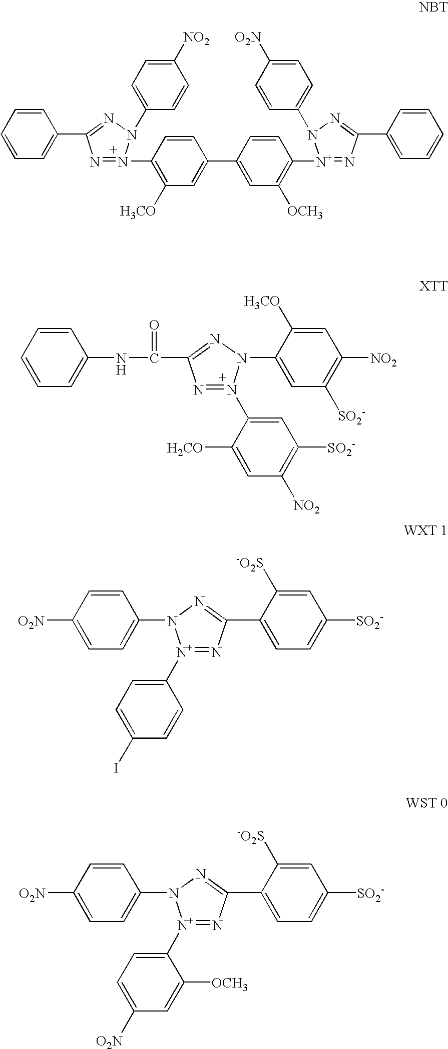

rat liver and heart homogenates. Thus, although there is no perfect assay, the method described herein using WST-1 is the most preferred as it overcomes many problems associated with conventional methods. It may be employed using a flow injection assay system for more throughput (e.g., 30 samples/hour) (Ukeda, supra)

### Testing of ATN-224 and Other Compositions on Cells In vitro

A. Assay for EC Migration

For EC migration, transwells are coated with type I collagen (50 μg/mL) by adding 200 μL of the collagen solution per transwell, then incubating overnight at 37° C. The transwells are assembled in a 24-well plate and a chemoattractant (e.g., FGF-2) is added to the bottom chamber in a total volume of 0.8 mL media. ECs, such as human umbilical vein ECs (HUVEC), which have been detached from monolayer culture using trypsin, are diluted to a final concentration of about 10⁶ cells/mL with serum-free media and 0.2 mL of this cell suspension is added to the upper chamber of each transwell. Inhibitors to be tested are added to both the upper and lower chambers, and the migration is allowed to proceed for 5 hrs in a humidified atmosphere at 37° C. The transwells are removed from the plate stained using DiffQuik®. Cells which did not migrate are removed from the upper chamber by scraping with a cotton swab and the membranes are detached, mounted on slides, and counted under a high-power field (400×) to determine the number of cells migrated. B. Biological Assay of Anti-Invasive Activity

The compositions of the invention are tested for their anti-invasive capacity. The ability of cells such as ECs or tumor cells (e.g., PC-3 human prostatic carcinoma) cells to invade through a reconstituted basement membrane (Matrigel®) in an assay known as a Matrigel® invasion assay system as described in detail by Kleinman et al., Biochemistry 25: 312-318, 1986 and Parish et al., Int. J. Cancer 52:378-383, 1992. Matrigel® is a reconstituted basement membrane containing type IV collagen, laminin, heparan sulfate proteoglycans such as perlecan, which bind to and localize bFGF, vitronectin as well as transforming growth factor-β (TGFβ), urokinase-type plasminogen activator (uPA), tissue plasminogen activator (tPA), and the serpin known as plasminogen activator inhibitor type 1 (PAI-1) (Chambers et al., Canc. Res. 55:1578-1585, 1995). It is accepted in the art that results obtained in this assay for compounds which target extracellular receptors or enzymes are predictive of the efficacy of these compounds in vivo (Rabbani et al., Int. J. Cancer 63: 840-845, 1995).

Such assays employ transwell tissue culture inserts. Invasive cells are defined as cells which are able to traverse through the Matrigel® and upper aspect of a polycarbonate membrane and adhere to the bottom of the membrane. Transwells (Costar) containing polycarbonate membranes (8.0 μm pore size) are coated with Matrigel® (Collaborative Research), which has been diluted in sterile PBS to a final concentration of 75 μg/mL (60 μL of diluted Matrigel® per insert), and placed in the wells of a 24-well plate. The membranes are dried overnight in a biological safety cabinet, then rehydrated by adding 100 μL of DMEM containing antibiotics for 1 hour on a shaker table. The DMEM is removed from each insert by aspiration and 0.8 mL of DMEM/10% FBS/antibiotics is added to each well of the 24-well plate such that it surrounds the outside of the transwell (“lower chamber”). Fresh DMEM/ antibiotics (100 μL), human Glu-plasminogen (5 μg/mL), and any inhibitors to be tested are added to the top, inside of the transwell (“upper chamber”). The cells which are to be tested are trypsinized and resuspended in DMEM/antibiotics, then added to the top chamber of the transwell at a final concentration of 800,000 cells/mL. The final volume of the upper chamber is adjusted to 200 μL. The assembled plate is then incubated in a humid 5% CO₂ atmosphere for 72 hours. After incubation, the cells are fixed and stained using DiffQuik® (Giemsa stain) and the upper chamber is then scraped using a cotton swab to remove the Matrigel® and any cells which did not invade through the membrane. The membranes are detached from the transwell using an X-acto® blade, mounted on slides using Permount® and cover-slips, then counted under a high-powered (400×) field. An average of the cells invaded is determined from 5-10 fields counted and plotted as a function of inhibitor concentration.

C. Tube-Formation Assays of Anti-Angiogenic Activity

The compounds of this invention are tested for their anti-angiogenic activity in one of two different assay systems in vitro.

Endothelial cells, for example, human umbilical vein ECs (HUVEC) or human microvascular ECs (HMVEC) which can be prepared or obtained commercially, are mixed at a concentration of 2×10⁵ cells/mL with fibrinogen (5 mg/mL in phosphate buffered saline (PBS) in a 1:1 (v/v) ratio. Thrombin is added (5 units/mL final concentration) and the mixture is immediately transferred to a 24-well plate (0.5 mL per well). The fibrin gel is allowed to form and then VEGF and bFGF are added to the wells (each at 5 ng/mL final concentration) along with the test compound. The cells are incubated at 37° C. in 5% CO₂ for 4 days at which time the cells in each well are counted and classified as either rounded, elongated with no branches, elongated with one branch, or elongated with 2 or more branches. Results are expressed as the average of 5 different wells for each concentration of compound. Typically, in the presence of angiogenic inhibitors, cells remain either rounded or form undifferentiated tubes (e.g. 0 or 1 branch).

This assay is recognized in the art to be predictive of angiogenic (or anti-angiogenic) efficacy in vivo (Min, H Y et al., Cancer Res. 56: 2428-2433, 1996).

In an alternate assay, EC tube formation is observed when ECs are cultured on Matrigel® (Schnaper et al., J. Cell. Physiol. 165:107-118 1995). Endothelial cells (1×10⁴ cells/well) are transferred onto Matrigel®-coated 24-well plates, and tube formation is quantitated after 48 hrs. Inhibitors are tested by adding them either at the same time as the ECs or at various time points thereafter. Tube formation can also be stimulated by adding (a) angiogenic growth factors such as bFGF or VEGF, (b) differentiation stimulating agents (e.g., PMA) or (c) a combination of these.

This assay models angiogenesis by presenting to the ECs a particular type of basement membrane, namely the layer of matrix which migrating and differentiating ECs might be expected to first encounter. In addition to bound growth factors, the matrix components found in Matrigel® (and in basement membranes in situ) or proteolytic products thereof may also be stimulatory for EC tube formation which makes this model complementary to the fibrin gel angiogenesis model previously described (Blood et al., Biochim. Biophys. Acta 1032:89-118, 1990; Odedra et al., Pharmac. Ther. 49:111-124, 1991). The compounds of this invention inhibit EC tube formation in both assays, which suggests that the compounds will also have anti-angiogenic activity.

D. Assays for the Inhibition of Proliferation

The ability of the compounds of the invention to inhibit the proliferation of EC's may be determined in a 96-well format. Type I collagen (gelatin) is used to coat the wells of the plate (0.1-1 mg/mL in PBS, 0.1 mL per well for 30 minutes at room temperature). After washing the plate (3× w/PBS), 3-6,000 cells are plated per well and allowed to attach for 4 hrs (37° C./5% CO₂) in Endothelial Growth Medium (EGM; Clonetics) or M199 medium containing 0.1-2% FBS. The medium and any unattached cells are removed at the end of 4 hrs and fresh media containing bFGF (1-10 ng/mL) or VEGF (1-10 ng/mL) is added to each well. Compounds to be tested are added last and the plate is allowed to incubate (37° C./5% CO₂) for 24-48 hrs. MTS (Promega) is added to each well and allowed to incubate from 1-4 hrs. The absorbance at 490 nm, which is proportional to the cell number, is then measured to determine the differences in proliferation between control wells and those containing test compounds.

Alternatively, cells at a density of 6000/well are plated in wells of 48 well microplates on 0.1% gelatin in 200 μl M200/2% FCS, and incubated at 37° C. in a humid atmosphere of 5% CO₂ for 16 hrs. Compounds to be tested are diluted in M200 supplemented with 2% FCS and 1 ng/ml FGF-2 and added to the cells. Positive control contain no compound, and negative controls contain no compound or FGF-2. Cells are incubated at 37° C./5% CO₂ for 72 hours. Cells are enumerated indirectly using the acid phosphatase method (Connolly, D T et al., 1986, Anal. Biochem 152:136-140). After removal of growth medium, the cells are lysed in buffer containing Triton X-100. The chromogenic substrate for acid phosphatase, p-nitrophenyl phosphate is added at a concentration of 100 mM. After incubation for 75 min. at 37° C., the reaction is stopped with 1N NaOH, and color is measured using a multiwell microplate reader.

A similar assay system can be set up with cultured adherent tumor cells. However, collagen may be omitted in this format. Tumor cells (e.g., 3,000-10,000/well) are plated and allowed to attach overnight. Serum free medium is then added to the wells, and the cells are synchronized for 24 hrs. Medium containing 10% FBS is then added to each well to stimulate proliferation. Compounds to be tested are included in some of the wells. After 24 hrs, MTS is added to the plate and the assay developed and read as described above.

E. Assays of Cytotoxicity

The anti-proliferative and cytotoxic effects of the compositions may be determined for various cell types including tumor cells, ECs, fibroblasts and macrophages. Anti-proliferative effects would be expected against tumor cells and stimulated ECs but, under some circumstances not quiescent ECs or normal human dermal fibroblasts. Any anti-proliferative or cytotoxic effects observed in the normal cells would represent non-specific toxicity of the agent

A typical assay would involve plating cells at a density of 5-10,000 cells per well in a 96-well plate. The compound to be tested is added at a concentration 10× the IC₅₀ measured in a binding assay (this will vary depending on the conjugate) and allowed to incubate with the cells for 30 minutes. The cells are washed 3× with media, then fresh media containing [³H]thymidine (1 μCi/mL) is added to the cells and they are allowed to incubate at 37° C. in 5% CO₂ for 24 and 48 hours. Cells are lysed at the various time points using 1 M NaOH and counts per well determined using a β-counter. Proliferation may be measured non-radioactively using MTS reagent or CyQuant® to measure total cell number. For cytotoxicity assays (measuring cell lysis), a Promega 96-well cytotoxicity kit is used. If there is evidence of anti-proliferative activity, induction of apoptosis may be measured using TumorTACS (Genzyme).

### Testing of ATN-224 and Other Compositions on Cells In Vivo

A. Corneal Angiogenesis Model

The protocol used is essentially identical to that described by Volpert et al. (1996, J. Clin. Invest. 98:671-679). Briefly, female Fischer rats (120-140 gms) are anesthetized and pellets (5 μl) comprised of Hydron®, bFGF (150 nM), and the compounds to be tested are implanted into tiny incisions made in the cornea 1.0-1.5 mm from the limbus. Neovascularization is assessed at 5 and 7 days after implantation. On day 7, animals are anesthetized and infused with a dye such as colloidal carbon to stain the vessels. The animals are then euthanized, the corneas fixed with formalin, and the corneas flattened and photographed to assess the degree of neovascularization. Neovessels may be quantitated by imaging the total vessel area or length or simply by counting vessels.

B. Matrigel® Plug Assay

This assay is performed essentially as described by Passaniti et al. (Lab Invest. 67:519-528 (1992). Ice-cold Matrigel® (e.g., 500 μL) (Collaborative Biomedical Products, Inc., Bedford, Mass.) is mixed with heparin (e.g., 50 μg/ml), FGF-2 (e.g., 400 ng/ml) and the compound to be tested. In some assays, bFGF may be substituted with tumor cells as the angiogenic stimulus. The Matrigel® mixture is injected subcutaneously into 4-8 week-old athymic nude mice at sites near the abdominal midline, preferably 3 injections per mouse. The injected Matrigel® forms a palpable solid gel. Injection sites are chosen such that each animal receives a positive control plug (such as FGF-2+heparin), a negative control plug (e.g., buffer+heparin) and a plug that includes the compound being tested for its effect on angiogenesis, e.g., (FGF-2+heparin+compound). All treatments are preferably run in triplicate. Animals are sacrificed by cervical dislocation at about 7 days post injection or another time that may be optimal for observing angiogenesis. The mouse skin is detached along the abdominal midline, and the Matrigel® plugs are recovered and scanned immediately at high resolution. Plugs are then dispersed in water and incubated at 37° C. overnight. Hemoglobin (Hb) levels are determined using Drabkin's solution (e.g., obtained from Sigma) according to the manufacturers' instructions. The amount of Hb in the plug is an indirect measure of angiogenesis as it reflects the amount of blood in the sample. In addition, or alternatively, animals may be injected prior to sacrifice with a 0.1 ml buffer (preferably PBS) containing a high molecular weight dextran to which is conjugated a fluorophore. The amount of fluorescence in the dispersed plug, determined fluorimetrically, also serves as a measure of angiogenesis in the plug. Staining with mAb anti-CD31 (CD31 is “platelet-EC adhesion molecule or PECAM”) may also be used to confirm neovessel formation and microvessel density in the plugs.

C. Chick Chorioallantoic Membrane (CAM) Angiogenesis Assay

This assay is performed essentially as described by Nguyen et al. (Microvascular Res. 47:31-40 (1994)). A mesh containing either angiogenic factors (bFGF) or tumor cells plus inhibitors is placed onto the CAM of an 8-day old chick embryo and the CAM observed for 3-9 days after implantation of the sample. Angiogenesis is quantitated by determining the percentage of squares in the mesh which contain blood vessels.

D. In Vivo Assessment Angiogenesis Inhibition and Anti-Tumor Effects Using the Matrigel® Plug Assay with Tumor Cells

In this assay, tumor cells, for example 1-5×10⁶ cells of the 3LL Lewis lung carcinoma or the rat prostate cell line MatLyLu, are mixed with Matrigel® and then injected into the flank of a mouse following the protocol described in Sec. B., above. A mass of tumor cells and a powerful angiogenic response can be observed in the plugs after about 5 to 7 days. The anti-tumor and anti-angiogenic action of a compound in an actual tumor environment can be evaluated by including it in the plug. Measurement is then made of tumor weight, Hb levels or fluorescence levels (of a dextran-fluorophore conjugate injected prior to sacrifice). To measure Hb or fluorescence, the plugs are first homogenize with a tissue homogenizer.

E. Xenograft Model of Subcutaneous (s.c.) Tumor Growth

Nude mice are inoculated with MDA-MB-231 cells (human breast carcinoma) and Matrigel® (1×10⁶ cells in 0.2 mL) s.c. in the right flank of the animals. The tumors are staged to 200 mm³ and then treatment with a test composition is initiated (100 μg/animal/day given q.d. IP). Tumor volumes are obtained every other day and the animals are sacrificed after 2 weeks of treatment. The tumors are excised, weighed and paraffin embedded. Histological sections of the tumors are analyzed by H and E, anti-CD31, Ki-67, TUNEL, and CD68 staining.

F. Xenograft Model of Metastasis

The compounds of this invention are also tested for inhibition of late metastasis using an experimental metastasis model (Crowley, C. W. et al., Proc. Natl. Acad. Sci. USA 90 5021-5025 (1993)). Late metastasis involves the steps of attachment and extravasation of tumor cells, local invasion, seeding, proliferation and angiogenesis. Human prostatic carcinoma cells (PC-3) transfected with a reporter gene, preferably the green fluorescent protein (GFP) gene, but as an alternative with a gene encoding the enzymes chloramphenicol acetyl-transferase (CAT), luciferase or LacZ, are inoculated into nude mice. This approach permits utilization of either of these markers (fluorescence detection of GFP or histochemical colorimetric detection of enzymatic activity) to follow the fate of these cells. Cells are injected, preferably iv, and metastases identified after about 14 days, particularly in the lungs but also in regional lymph nodes, femurs and brain. This mimics the organ tropism of naturally occurring metastases in human prostate cancer. For example, GFP-expressing PC-3 cells (1×10⁶ cells per mouse) are injected iv into the tail veins of nude (nu/nu) mice. Animals are treated with a test composition at 100 μg/animal/day given q.d. IP. Single metastatic cells and foci are visualized and quantitated by fluorescence microscopy or light microscopic histochemistry or by grinding the tissue and quantitative colorimetric assay of the detectable label.

G. Inhibition of Spontaneous Metastasis In Vivo by HPRG and Functional Derivatives

The rat syngeneic breast cancer system (Xing et al., Int. J. Cancer 67:423-429 (1996) employs Mat BIII rat breast cancer cells. Tumor cells, for example about 10⁶ suspended in 0.1 mL PBS, are inoculated into the mammary fat pads of female Fisher rats. At the time of inoculation, a 14-day Alza osmotic mini-pump is implanted intraperitoneally to dispense the test compound. The compound is dissolved in PBS (e.g., 200 mM stock), sterile filtered and placed in the minipump to achieve a release rate of about 4 mg/kg/day. Control animals receive vehicle (PBS) alone or a vehicle control peptide in the minipump. Animals are sacrificed at about day 14.
Therapeutic Outcomes

In the rats treated with the active compounds of the present invention, significant reductions in the size of the primary tumor and in the number of metastases in the spleen, lungs, liver, kidney and lymph nodes (enumerated as discrete foci) are observed. Histological and immunohistochemical analysis reveal increased necrosis and signs of apoptosis in tumors in treated animals. Large necrotic areas are seen in tumor regions lacking neovascularization.

H. 3LL Lewis Lung Carcinoma: Primary Tumor Growth

This tumor line arose spontaneously in 1951 as carcinoma of the lung in a C57BL/6 mouse (Cancer Res 15:39, 1955. See, also Malave, I. et al., J. Nat'l. Canc. Inst. 62:83-88 (1979)). It is propogated by passage in C57BL/6 mice by subcutaneous (sc) inoculation and is tested in semiallogeneic C57BL/6×DBA/2 F₁ mice or in allogeneic C3H mice. Typically six animals per group for subcutaneously (sc) implant, or ten for intramuscular (im) implant are used. Tumor may be implanted sc as a 2-4 mm fragment, or im or sc as an inoculum of suspended cells of about 0.5-2×10⁶-cells. Treatment begins 24 hours after implant or is delayed until a tumor of specified size (usually approximately 400 mg) can be palpated. The test compound is administered ip daily for 11 (??) days

Animals are followed by weighing, palpation, and measurement of tumor size. Typical tumor weight in untreated control recipients on day 12 after im inoculation is 500-2500 mg. Typical median survival time is 18-28 days. A positive control compound, for example cyclophosphamide at 20 mg/kg/injection per day on days 1-11 is used. Results computed include mean animal weight, tumor size, tumor weight, survival time. For confirmed therapeutic activity, the test composition should be tested in two multi-dose assays.

I. 3LL Lewis Lung Carcinoma: Primary Growth and Metastasis Model

This model has been utilized by a number of investigators. See, for example, Gorelik, E. et al., J. Nat'l. Canc. Inst. 65:1257-1264 (1980); Gorelik, E. et al., Rec. Results Canc. Res. 75:20-28 (1980); Isakov, N. et al., Invasion Metas. 2:12-32 (1982); Talmadge J. E. et al., J. Nat'l. Canc. Inst. 69:975-980 (1982); Hilgard, P. et al., Br. J. Cancer 35:78-86(1977)). Test mice are male C57BL/6 mice, 2-3 months old. Following sc, im, or intra-footpad implantation, this tumor produces metastases, preferentially in the lungs. With some lines of the tumor, the primary tumor exerts anti-metastatic effects and must first be excised before study of the metastatic phase (see also U.S. Pat. No. 5,639,725).

Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected subcutaneously, either in the dorsal region or into one hind foot pad of C57BL/6 mice. Visible tumors appear after 3-4 days after dorsal sc injection of 10⁶ cells. The day of tumor appearance and the diameters of established tumors are measured by caliper every two days.

The treatment is given as one or two doses of peptide or derivative, per week. In another embodiment, the peptide is delivered by osmotic minipump.

In experiments involving tumor excision of dorsal tumors, when tumors reach about 1500 mm³ in size, mice are randomized into two groups: (1) primary tumor is completely excised; or (2) sham surgery is performed and the tumor is left intact. Although tumors from 500-3000 mm³ inhibit growth of metastases, 1500 mm³ is the largest size primary tumor that can be safely resected with high survival and without local regrowth. After 21 days, all mice are sacrificed and autopsied.

Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ¹²⁵IdUrd into lung cells (Thakur, M. L. et al., J. Lab. Clin. Med. 89:217-228 (1977). Ten days following tumor amputation, 25 μg of fluorodeoxyuridine is inoculated into the peritoneums of tumor-bearing (and, if used, tumor-resected mice). After 30 min, mice are given 1 μCi of ¹²⁵IdUrd (iododeoxyuridine). One day later, lungs and spleens are removed and weighed, and a degree of ¹²⁵IdUrd incorporation is measured using a gamma counter.

In mice with footpad tumors, when tumors reach about 8-10 mm in diameter, mice are randomized into two groups: (1) legs with tumors are amputated after ligation above the knee joints; or (2) mice are left intact as nonamputated tumor-bearing controls. (Amputation of a tumor-free leg in a tumor-bearing mouse has no known effect on subsequent metastasis, ruling out possible effects of anesthesia, stress or surgery). Mice are killed 10-14 days after amputation. Metastases are evaluated as described above.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of larger doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 10⁶ 3LL cells. Amputation of tumors produced following inoculation of 10⁵ tumor cells dramatically accelerated metastatic growth. These results were in accord with the survival of mice after excision of local tumors. The phenomenon of acceleration of metastatic growth following excision of local tumors had been repeatedly observed (for example, see U.S. Pat. No. 5,639,725). These observations have implications for the prognosis of patients who undergo cancer surgery.

### Pharmaceutical Compositions and Their Administration

ATN-224 is the active agent in the present pharmaceutical compositions. As used herein, the term the “active agent” be active per se, or may act as a “pro-drug” that is converted in vivo to the active form. ATN-224 may be incorporated into convenient dosage forms, such as capsules, impregnated wafers, tablets or injectable preparations. Solid or liquid pharmaceutically acceptable carriers may be employed.

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Remington's Pharmaceutical Sciences, Mack Publishing Company, Easton Pa. (latest edition). The pharmaceutical preparations are made following conventional techniques of pharmaceutical chemistry involving such steps as mixing, granulating and compressing, when necessary for tablet forms, or mixing, filling and dissolving the ingredients, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

The present invention may be used in the treatment of any of a number of animal genera and species, and are equally applicable in the practice of human or veterinary medicine. Thus, the pharmaceutical compositions can be used to treat domestic and commercial animals, including birds and more preferably mammals, in particular, humans.

The term “systemic administration” refers to administration in a manner that results in the introduction of the composition into the subject's circulatory system or otherwise permits its spread throughout the body, such as intravenous (i.v.) injection or infusion. “Regional” administration refers to administration into a specific, and somewhat more limited, anatomical space, such as intraperitoneal, intrathecal, subdural, or to a specific organ. Examples include intravaginal, intrapenile, intranasal, intrabronchial(or lung instillation), intracranial, intra-aural or intraocular. The term “local administration” refers to administration of a composition or drug into a limited, or circumscribed, anatomic space, such as intratumoral injection into a tumor mass, subcutaneous (s.c.) injections, intramuscular (i.m.) injections. One of skill in the art would understand that local administration or regional administration often also result in entry of a composition into the circulatory system, i.e.,, so that s.c. or i.m. are also routes for systemic administration. Injectable or infusible preparations can be prepared in conventional forms, either as solutions or suspensions, solid forms suitable for solution or suspension in liquid prior to injection or infusion, or as emulsions. Though the preferred routes of administration are systemic, such as i.v., the pharmaceutical composition may be administered topically or transdermally, orally or rectally.

For topical application, the compound may be incorporated into topically applied vehicles such as a salve or ointment. The carrier for the active ingredient may be either in sprayable or nonsprayable form. Non-sprayable forms can be semi-solid or solid forms comprising a carrier indigenous to topical application and having a dynamic viscosity preferably greater than that of water. Suitable formulations include, but are not limited to, solution, suspensions, emulsions, creams, ointments, powders, liniments, salves, and the like. If desired, these may be sterilized or mixed with auxiliary agents, e.g., preservatives, stabilizers, wetting agents, buffers, or salts for influencing osmotic pressure and the like. Preferred vehicles for non-sprayable topical preparations include ointment bases, e.g., polyethylene glycol-1000 (PEG-1000); conventional creams such as HEB cream; gels; as well as petroleum jelly and the like.

Also suitable for topic application as well as for lung instillation are sprayable aerosol preparations wherein the compound, preferably in combination with a solid or liquid inert carrier material, is packaged in a squeeze bottle or in admixture with a pressurized volatile, normally gaseous propellant. The aerosol preparations can contain solvents, buffers, surfactants, perfumes, and/or antioxidants in addition to the compounds of the invention.

For the preferred topical applications, especially for humans, it is preferred to administer an effective amount of the compound to an affected area, e.g., skin surface, mucous membrane, eyes, etc. This amount will generally range from about 0.001 mg to about 1 g per application, depending upon the area to be treated, the severity of the symptoms, and the nature of the topical vehicle employed.

Therapeutic compositions for treating tumors and cancer may comprise, in addition to ATN-224, one or more additional anti-tumor agents, such as mitotic inhibitors, e.g., vinblastine; alkylating agents, e.g., cyclophosphamide; folate inhibitors, e.g., methotrexate, piritrexim or trimetrexate; antimetabolites, e.g., 5-fluorouracil and cytosine arabinoside; intercalating antibiotics, e.g., adriamycin and bleomycin; enzymes or enzyme inhibitors, e.g., asparaginase, topoisomerase inhibitors such as etoposide; or biological response modifiers, e.g., interferons or interleukins. In fact, pharmaceutical compositions comprising any known cancer therapeutic in combination with ATN-224 are within the scope of this invention. The pharmaceutical composition may also comprise one or more other medicaments to treat additional symptoms for which the target patients are at risk, for example, anti-infectives including antibacterial, anti-fungal, anti-parasitic, anti-viral, and anti-coccidial agents.

The therapeutic dosage administered is an amount which is therapeutically effective, as is known to or readily ascertainable by those skilled in the art. The dose is also dependent upon the age, health, and weight of the recipient, kind of concurrent treatment(s), if any, the frequency of treatment, and the nature of the effect desired, such as, for example, anti-inflammatory effects or anti-bacterial effect.

### Therapeutic Methods

The methods of this invention may be used to inhibit tumor growth and invasion in a subject or to suppress angiogenesis induced by tumors by inhibiting EC growth or viability, in addition to effects on tumor cell viability. By inhibiting the growth or invasion of a tumor or angiogenesis, the methods result in inhibition of tumor metastasis. A vertebrate subject, preferably a mammal, more preferably a human, is administered an amount of the ATN-224 to inhibit tumor growth, invasion or angiogenesis.

Doses of ATN-224 preferably includes pharmaceutical dosage units comprising an effective amount of the compound. Dosage unit form refers to physically discrete units suited as unitary dosages for a mammalian subject; each unit contains a predetermined quantity of active material calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects.

Other copper binding compounds that inhibit SOD1 activity, endothelial cell proliferation and migration and angiogenesis, identified and/or designed in accordance with the present invention, are also useful as therapeutic agents. Pharmaceutical compositions comprising such compounds are included in the scope of this invention.

By an effective amount is meant an amount sufficient to achieve a steady state concentration in vivo which results in a measurable reduction in any relevant parameter of disease or surrogate marker which may include growth of primary or metastatic tumor, any accepted index of angiogenic activity, or a measurable prolongation of disease-free interval or of survival. For example, a reduction in tumor growth in 20% of patients is considered efficacious (Frei III, E., The Cancer Journal 3:127-136 (1997)). However, an effect of this magnitude is not considered to be a minimal requirement for the dose to be effective in accordance with this invention.

In one embodiment, an effective dose is preferably 10-fold and more preferably 100-fold higher than the 50% effective dose (ED₅₀) of the compound in an in vivo assay as described herein.

The amount of the active compound to be administered depends on the disease or condition, the route of administration, the health and weight of the recipient, the existence of other concurrent treatment, if any, the frequency of treatment, the nature of the effect desired, for example, inhibition of tumor metastasis, and the judgment of the skilled practitioner.

A preferred daily dose for treating a subject, preferably mammalian, more preferably human, with a tumor is an amount of between about 0.1 mg/kg body weight and about 10 mg/kg body weight. A typical human single dosage is between about 90 and about 500 mg, and is preferably given orally. Due to the lack of toxicity of this agent, such a treatment regimen can be continued for weeks, months or even longer.

For topical administration, dosages in the range of about 0.01-20% concentration (by weight) of the compound, preferably 1-5%, are suggested. The foregoing ranges are, however, suggestive, as the number of variables in an individual treatment regime is large, and considerable excursions from these preferred values are expected.

An effective amount or dose for inhibiting EC proliferation in vitro is in the range of about 1 picogram to about 0.5 mg per cell. Effective doses and optimal dose ranges may be determined in vitro using the methods described herein.

The compound of the invention may be characterized as producing an inhibitory effect on tumor cell or EC proliferation or on angiogenesis, on tumor metastasis or on inflammatory reactions to a tumor. The compounds are especially useful in producing an anti-tumor effect in a mammalian host, preferably human, harboring a tumor.

Angiogenesis inhibitors may play a role in preventing inflammatory angiogenesis and gliosis following traumatic spinal cord injury, thereby promoting the reestablishment of neuronal connectivity (Wamil, A. W. et al., Proc. Nat'l. Acad. Sci. USA 95:13188-13193 (1998)). Therefore, ATN-224 may be administered as soon as possible after traumatic spinal cord injury and continued for several days up to about four weeks thereafter to inhibit the angiogenesis and gliosis that would sterically prevent reestablishment of neuronal connectivity. The treatment reduces the area of damage at the site of spinal cord injury and facilitates regeneration of neuronal function and thereby prevents paralysis. Such treatment is expected also to protect axons from Wallerian degeneration, reverse aminobutyrate-mediated depolarization (occurring in traumatized neurons), and improve recovery of neuronal conductivity of isolated central nervous system cells and tissue in culture.

Having now generally described the invention, the same will be more readily understood through reference to the following examples which are provided by way of illustration, and are not intended to be limiting of the present invention, unless specified.

### EXAMPLE I

### Materials and Methods

### Cell Culture

Cultures of HUVEC cultures were maintained in M200/lsgs media (Cascade Biologicals, Portland Oreg.) on 0.1% gelatin. See: Antoniv et al. 2001, J. Biol. Chem., 276:21754-64)

### Proliferation Assays:

Cells at a density of 6000/well were plated in wells of 48 well microplates on 0.1% gelatin in 200 μl M200/2% FCS, and were incubated at 37° C. in a humid atmosphere of 5% CO₂ for 16 hrs. Compounds to be tested were diluted in M200 supplemented with 2% FCS and 1 ng/ml FGF-2 and added to the cells. Positive control contained no compound, and negative controls contained no compound or FGF-2. Cells were incubated at 37° C./5% CO₂ for 72 hours. Cells were enumerated indirectly using the acid phosphatase method. After removal of growth medium, the cells were lysed in buffer containing the detergent Triton X-100. The chromogenic substrate for acid phosphatase, p-nitrophenyl phosphate was added at a concentration of 100 mM. After incubation for 75 min. at 37° C., the reaction was stopped with 1N NaOH and color was measured using a multiwell microplate reader.

### Matrigel® Plug Assay

Matrigel® (500 mL) on ice was mixed with heparin (50 mg/ml), FGF-2 (800 ng/ml), VEGF (300 ng/ml) and the test compound. Positive control plugs did not include the test compound, and negative controls plugs did not contain the angiogenic factors. In some experiments, the test compound was given orally. The Matrigel® mixture was injected subcutaneously into multiple sites of 4-8 week-old female BALB/c/nude mice. Animals were sacrificed and the plugs recovered 5 days post-injection. The plugs were then minced and homogenized with a tissue homogenizer. Hemoglobin levels in the plugs were determined using Drabkin's solution according to the manufacturers' instructions (Sigma Chemicals).

### Cell Partitioning of ATN-224

HUVECs were plated on 0.1% gelatin at 2-4×10⁶ cells in T-25 flasks in M200 medium with 2% FCS. Cells were incubated at 37° C./5% CO₂ for 16 hrs. Cells were rinsed in M200/2% FCS and incubated for a further 2 hrs in medium to which ATN-224 and FGF-2 (1 ng/ml) were added. Cells were then rinsed 3× with PBS, and trypsinized. After centrifugation (220×g for minutes) cell pellets were lysed in 200 μL lysis buffer (10 mM HEPES pH 8.0, 1.5 mM MgCl₂, 10 mM KCl, 300 mM sucrose, 0.1% NP-40, Roche mini complete protease inhibitor at 1 tablet/10 mL, 0.5 mM PMSF). The molybdenum content of the lysate was analyzed by Inductively Coupled Plasma-Mass Spectrometry (ICP-MS) (ERI, Vancouver, BC, Canada).

### Assay of CuZn SOD (SOD1) Activity

SOD1 was assayed with a commercially available kit (Dojindo) based on the xanthine/xanthine oxidase superoxide generation reaction coupled to a water soluble tetrazolium salt, WST-1.

### ATN-224 Inhibition of SOD1 in vitro.

Standard curves (0-13 nM) of bovine SOD were generated using the SOD1 assay described above in the presence of increasing concentrations of ATN-224. Appropriate blanks comprising ATN-224 in the absence of SOD1 enzyme were also generated. The inhibition of SOD1 activity was calculated from these standard curves.

### Removal of Copper from SOD1

Bovine SOD1 (7.5 μM) was incubated with increasing concentrations of ATN-224 for 30 minutes at room temperature. The protein was then purified by gel filtration chromatography. The sample was then analyzed for molybdenum content and copper content by ICP-MS.

### ATN-224 Inhibits the Activity of Intracellular CuZnSOD.

HUVECs were plated on 0.1% gelatin at 2-4×10⁶ cells in T-25 flasks in M200 medium with 2% FCS. Cells were incubated at 37° C./5% CO₂ for 16 hrs. Cells were rinsed with M200/2% FCS and incubated for a further 2 hrs in medium to which varying concentration of ATN-224 and FGF-2 (at 1 ng/ml) were added. At various intervals, medium was removed and the cells lysed in lysis buffer. Lysates were assayed for SOD1 activity as described above. Lysates were also analyzed by Western blot using a SOD1-specific polyclonal antibody (Biodesign, ME). These experiments were repeated in increasing concentrations of serum.

### Visualization of Superoxide Using Dihydrethidine (DHE)

HUVECs were plated in 6 well cluster plates on 0.1% gelatin at 6000 cells/well in M200/2% FCS. Cells were incubated at 37° C./5% CO₂ for 16 hrs. Cells were rinsed, and ATN-224 was added in medium supplemented with 1 ng/ml FGF-2. Cells were incubated at 37 ° for 72 hours, rinsed in PBS, and DHE (Molecular probes) was added (5 μM) to the cells. Cells were then visualized microscopically using a fluorescent lamp and a red filter.

### Effects of Synthetic Porphyrin SOD Mimetic MnTBAP

HUVEC proliferation assays were performed as described. Increasing concentrations of ATN-224 was added to cultures in the presence or absence of 100 μM MnTBAP (AG Scientific, San Diego). Cell number was estimated using the acid phosphatase method described above.

Matrigel plug assays were performed as described above. Selected concentrations of ATN-224 and MnTBAP were added to the plug to evaluate inhibition of angiogenesis and its reversal.

### EXAMPLE II

### ATN-224 Inhibits Proliferation of Endothelial Cells

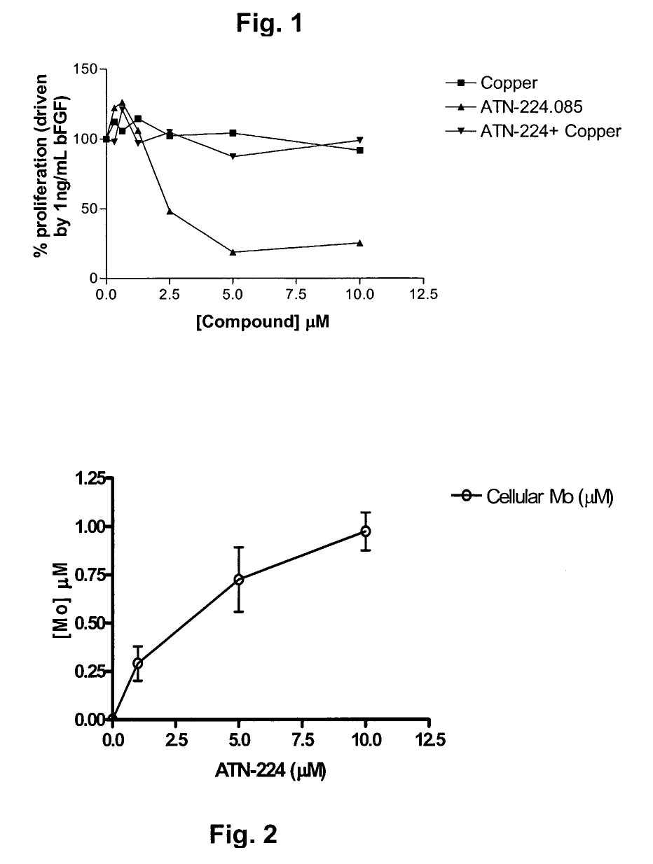

ATN-224 can inhibit the proliferation of HUVEC cells in a dose-dependent manner with an IC50 value of 1-2 μM (FIG. 1). Inhibition of FGF-2 driven proliferation can be completely abrogated by adding equimolar concentrations of copper to the assay (FIG. 1).

ATN-224 was found to be internalized by ECs. HUVECs were incubated with increasing, concentrations of ATN-224 for 2 hours at 37° C., and the cells were analyzed for Mo content by ICP-MS. There was a dose-dependent increase in the Mo concentration of cells (FIG. 2). Thus, ATN-224 binds to and accumulates in ECs.

### EXAMPLE III

### ATN-224 Inhibits the Activity of SOD1 In vitro

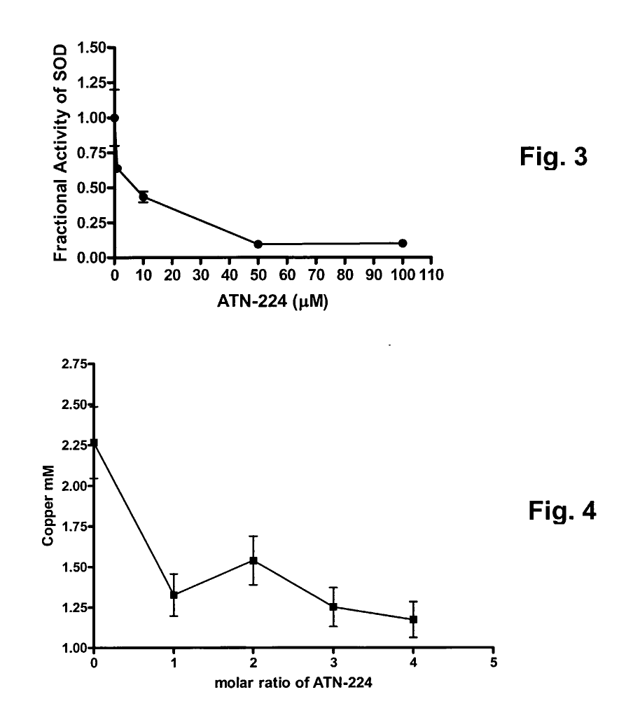

CuZnSOD (SOD1) is a copper-dependent enzyme which catalyzes the dismutation of superoxide ions to H₂O₂. ATN-224 inhibited this reaction in an in vitro enzyme assay (FIG. 3) utilizing xanthine/xanthine oxidase to generate the superoxide anions. ATN-224 did not act by inhibiting generation of superoxide ions in this assay.

Increasing concentrations of ATN-224 were incubated with bovine CuZnSOD for 20 min, and the protein was purified by gel filtration chromatography. The protein was analyzed for Mo and Cu content by ICP-MS. ATN-224 did not bind to CuZnSOD. An appreciable loss of copper was detected, indicating that ATN-224 removes copper from CuZnSOD (FIG. 4).

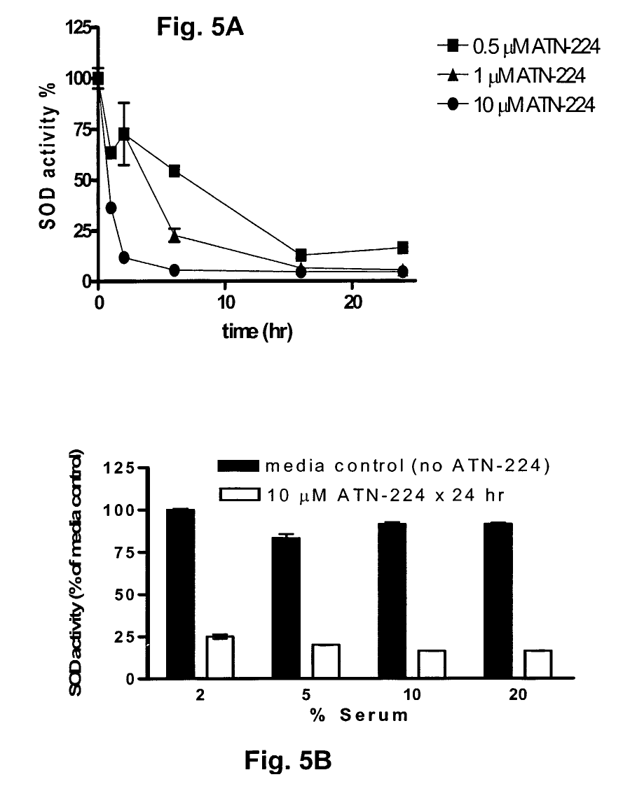

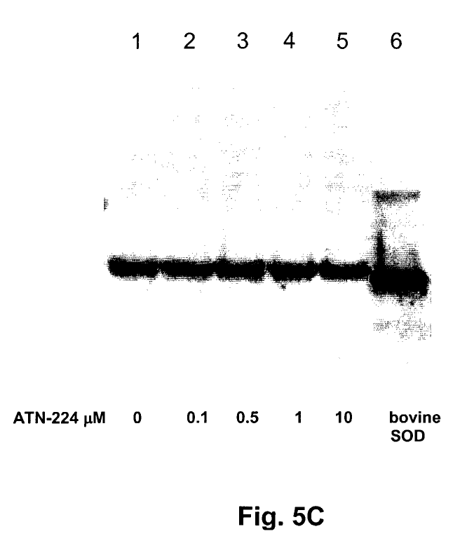

HUVECs were incubated with one of 3 concentrations of ATN-224 for various durations up to 24 hours. The cells were harvested at selected time points and assayed for SOD1 activity. FIG. 5a demonstrates the dose and time dependent inhibition of intracellular CuZnSOD by exogenously administered ATN-224. This inhibition was unaffected by the presence of serum (FIG. 5b), indicating that ATN-224 can partition from protein components in serum to the cell. Western blot analysis of cell lysates indicated that incubation of cells with ATN-224 did not affect the level of CuZnSOD protein (FIG. 5c).

### EXAMPLE IV

### ATN-224 Induces Increases in Superoxide Anions in HUVECs

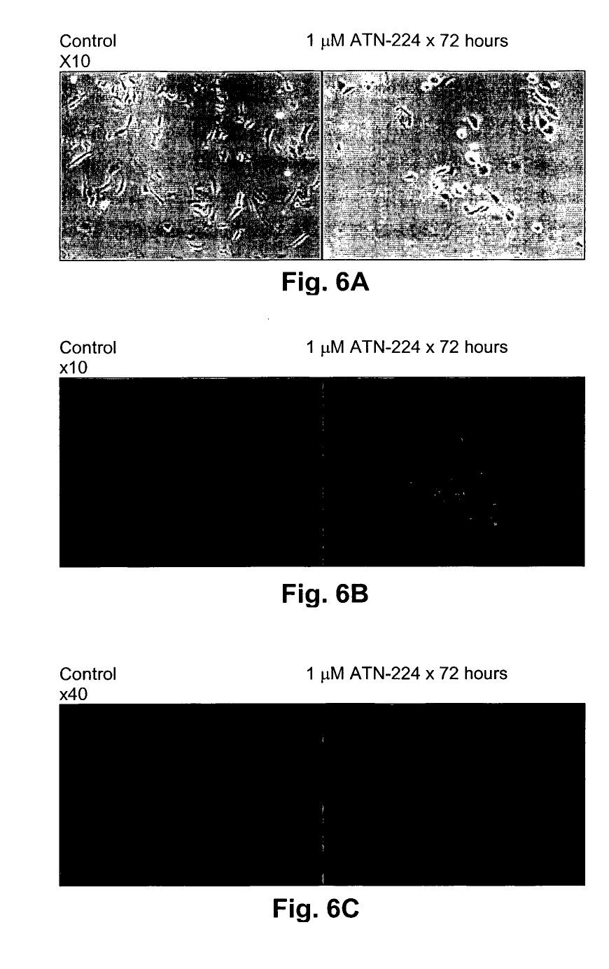

HUVECs were incubated with concentrations of ATN-224 equivalent to IC₅₀ values (for inhibition of enzyme activity). After 72 hours, cells were rinsed in PBS and incubated in fresh PBS containing 5 μM DHE. DHE reacts with superoxide ions to form a fluorescent product. After 72 hrs incubation, ATN-224-treated HUVECs showed strong fluorescence compared to control HUVECs (FIG. 6).

### EXAMPLE V

### Superoxide Dismutase Mimetics Can Abrogate the ATN-224 Mediated Inhibition of Endothelial Cell Proliferation

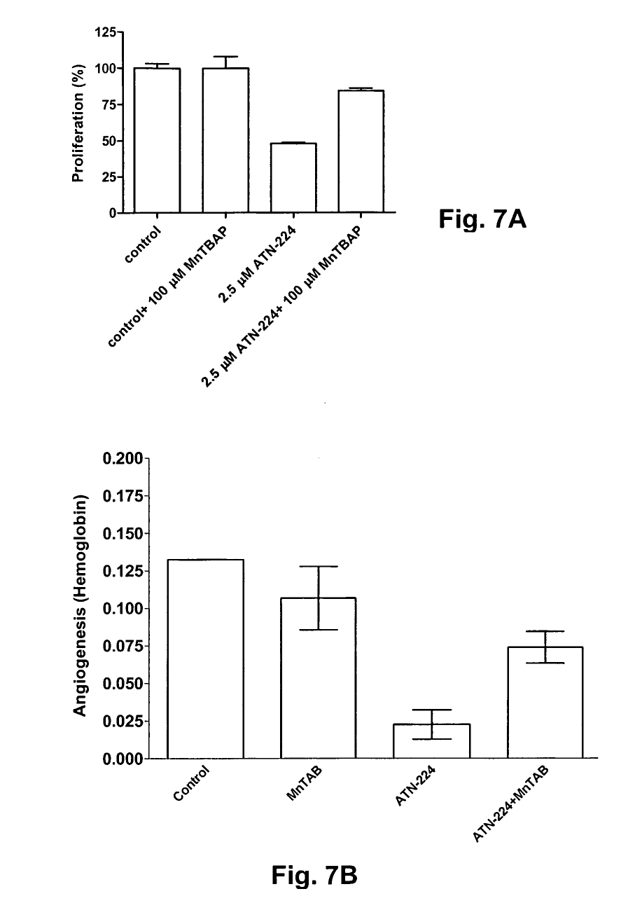

In an effort to determine whether the inhibition of CuZnSOD and the subsequent accumulation of superoxide ions plays a direct role in the inhibition of EC proliferation, cells were induced to proliferate in the presence of ATN-224 and the cell permeable SOD mimetic MnTBAP. MnTBAP is able to abrogate most of the inhibition of cell proliferation (FIG. 7) indicating that inhibition of CuZnSOD activity has a direct role in the anti-endothelial effects of ATN-224.

### EXAMPLE VI

### SOD Mimetics Can Abrogate the ATN-224-Mediated Inhibition of Angiogenesis

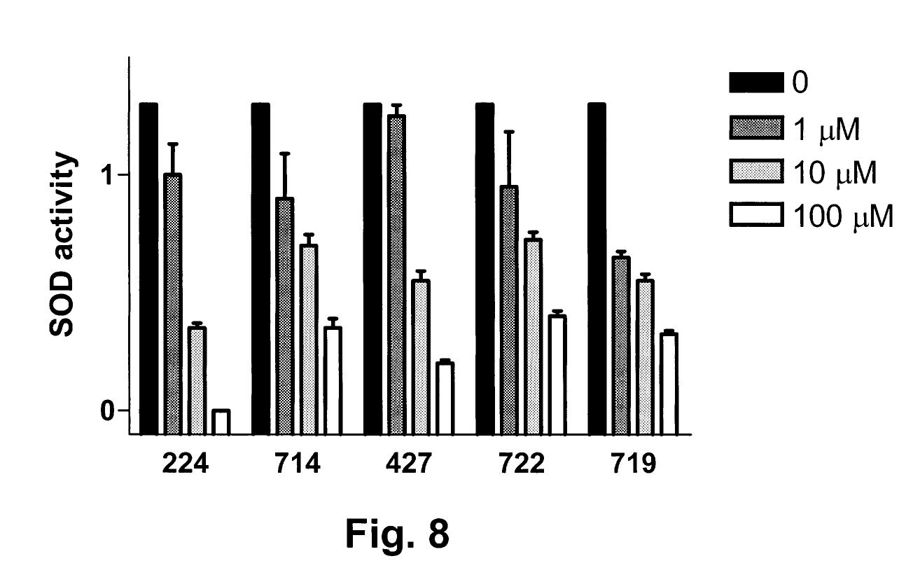

In an effort to determine whether the inhibition of CuZnSOD and the subsequent accumulation of superoxide ions plays a direct role in the inhibition of angiogenesis, growth factors embedded in Matrigel plugs were implanted in mice in the presence of ATN-224 and the cell permeable SOD mimetic MnTBAP. Normally, angiogenesis is locally stimulated in these plugs, which was inhibited by ATN-224. MnTBAP abrogated most of this inhibition (FIG. 8). It was concluded that the antiangiogenic effects of ATN-224 is influenced by inhibition of CuZnSOD activity.

### EXAMPLE VII

### Identification or Design of Molecules that Share Properties of ATN-224

Based on the inventors' knowledge that ATN-224 targets copper specifically, and thus targets SOD1 activity, it is possible to use the known chemical structure of ATN-224 to identify or design other compounds that share common structural features, and use the SOD assay in conjunction with the proliferation assay to screen such candidate compounds.

The sulfur-sulfur distance (3.66 Å) has been determined geometrically from Cu—Mo, Cu—S, and Mo—S distances from EXAFS (JACS (03) 125, 1704). Also, polythiaethers are Cu-selective with a S-S distance of 3.48 Å (Inorg. Chem. (99) 5906).

The present inventors have selected the following criteria and have searched commercially available database (Specs, ca. 235,000 structures) for small molecules that satisfy the physicochemical criteria described above. A representative set of 15 compounds was tested in the SOD1 assay and the EC proliferation assay as described herein. Results with three of these compounds (ATN-714, ATN-719 and ATN 722, shown in Table 1, below, plus choline tetrathiotungstate (ATN-427), a “3^{rd} generation” compound, in inhibiting SOD activity are shown in FIG. 8. ATN-224 is also shown as a positive control. Structures and molecular masses appear in a table below. All four of the above compounds had inhibited SOD1 activity.

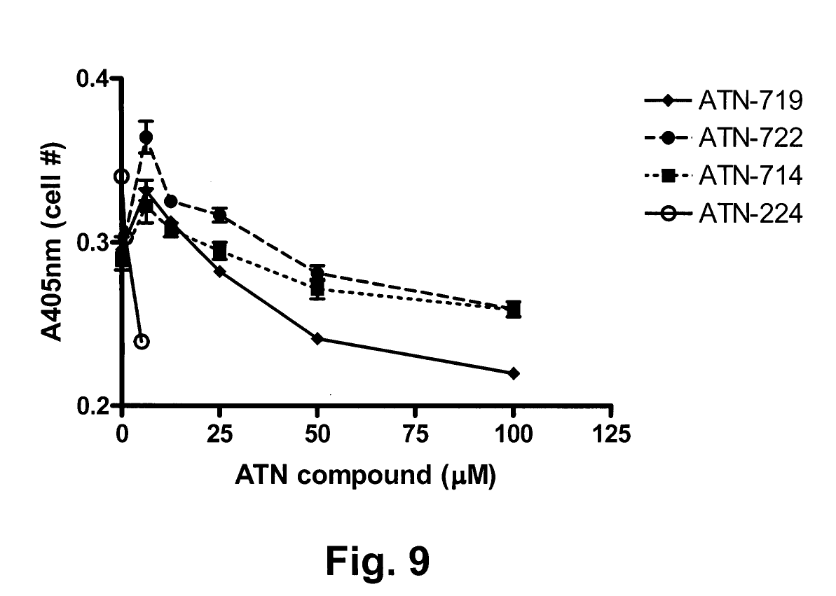

Three of these compounds were tested for their effect on EC proliferation. Results shown in FIG. 9, indicate that all three compounds showed dose-dependent inhibition of proliferation (in a colorimetric assay). Their activity was less potent that of ATN-224.

These results demonstrate that three compounds, identified using the criteria indicated above, inhibited SOD enzymatic activity and EC proliferation. Moreover, choline tetrathiotungstate was also an effective inhibitor of SOD1 activity.

The references cited above are all incorporated by reference in their entirety herein, whether specifically incorporated or not. Having now fully described this invention, it will be appreciated by those skilled in the art that the same can be performed within a wide range of equivalent parameters, concentrations, and conditions without departing from the spirit and scope of the invention and without undue experimentation.

TABLE 1

| ATN # | MW | Structure |
|---|---|---|
| 714 | 263.39 | 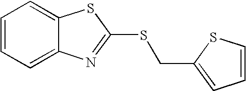 |
| 719 | 218.334 | 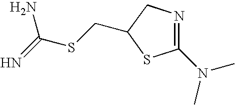 |
| 722 | 261.356 | 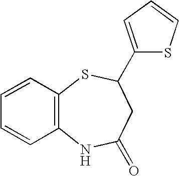 |

1. A method of identifying from among a plurality of existing compounds a molecule that is useful as a copper-binding angiogenesis inhibitor and/or anti-cancer agent, which method comprises:
(a) selecting a compound or a plurality of compounds with the following characteristics:
(i) a molecular mass of less than about 1000 Da,
(ii) at least two sulfur atoms separated from one another by 2 or 3 atoms, and
(iii) optionally, one or both of the sulfur atoms has a lone pair of electrons;
(b) testing said compound or plurality of compounds selected in (a) for their ability to remove copper from the CuZnSOD (SOD1) enzyme or to inhibit the catalytic activity of SOD1, and selecting one or more compounds with such copper-removing or enzyme-inhibitory activity; and
(c) optionally, testing said compound or compounds selected in (b) for their ability to inhibit cell proliferation in vitro,
wherein a compound that has said characteristics of (a), tests positive for SOD1-inhibitory activity and, optionally, inhibits cell proliferation in vitro, is identified as being useful as said angiogenesis inhibitor and/or anticancer agent.

2. The method of claim 1, wherein, the compound selected in selecting process (a) also has the ability to adopt a conformation that places the two sulfur atoms at a distance of between about 2 Å and about 5 Å.

3. The method of claim 2, wherein the compound selected in selecting process (a) has the ability to adopt a conformation that places the two sulfur atoms of a distance of between about 3.4 Å and about 3.8 Å.

4. The method of claim 1 wherein selecting process (a) is performed computationally.

5. The method of claim 1, wherein the compound or compounds are tested in step (c) for inhibition of the proliferation of activated endothelial cells.

6. The method of claim 1, wherein the compound or compounds are tested in step (c) for inhibition of the proliferation of tumor cells.

7. The method of claim 1 wherein the assay for SOD1 inhibitory activity is a biochemical assay.

8. The method of claim 7 wherein the biochemical assay comprises use of chromogenic water-soluble tetrazolium salts that yield a colored product.

9. The method of claim 8 wherein said tetrazolium salt is WST-1.

10. The method of claim 1, wherein the compound or compounds are further tested in an assay of endothelial cell migration.

11. The method of claim 1, wherein the compound or compounds are further tested in an assay of endothelial cell growth.

12. The method of claim 1, wherein the compound or compounds are further tested for tumor growth inhibition in an in vivo assay.

13. The method of claim 12 wherein the assay is a Matrigel® plug assay.

14. The method of claim 1, wherein said compound inhibits inhibition of SOD1, cell migration, cell growth and/or tumor growth by at least about 10%.

15. The method of claim 14 wherein said compound inhibits inhibition of SOD1, cell migration, cell growth and/or tumor growth by at least about 25%.

16. The method of claim 15 wherein said compound inhibits inhibition of SOD1, cell migration, cell growth and/or tumor growth by at least about 50%.

17. The method of claim 16 wherein said compound inhibits inhibition of SOD1, cell migration, cell growth and/or tumor growth by at least about 70%.

18. A method of designing a copper-binding molecule that removes copper from SOD1, thereby inhibiting SOD1 enzymatic activity, and is therefore useful as an antiangiogenic and/or anti-cancer agent, the method comprising determining atomic constituents and conformational parameters of the molecule being designed such that the molecule
(a) has the following physicochemical characteristics:
(i) a molecular mass of less than about 1000 Daltons
(ii) at least 2 sulfur atoms separated from one another by 2 or 3 atoms
(iii) optionally, one or both of the sulfur atoms has a lone pair of electrons;
(b) has the following biochemical characteristics:
(i) removes copper from SOD1; or
(ii) inhibits the catalytic activity of SOD1, and
(c) has one or more of the following effects on cells:
(i) inhibits proliferation of activated endothelial cells in vitro,
(ii) inhibits proliferation of tumor cells in vitro;
(iii) inhibits endothelial cell migration in vitro or in vivo;
(iv) inhibits tumor cell growth in vivo;
thereby designing said molecule.

19. The method of claim 18 wherein, the molecule being designed has the ability to adopt a conformation that places the two sulfur atoms at a distance of between about 2 Å and about 5 Å.

20. The method of claim 19 wherein, the molecule being designed has the ability to adopt a conformation that places the two sulfur atoms of a distance of between about 3.4 Å and 3.6 Å.

21. A method for making a copper-binding molecule that removes copper from SOD1, thereby inhibiting SOD1 enzymatic activity, and is therefore useful as an antiangiogenic and/or anti-cancer agent, which method comprises:
(a) designing the molecule in accordance with claim 18;
(b) selecting a synthetic process that will produce said molecule and stabilize its structure;
(c) employing the synthetic process of (b) to synthesize the molecule, thereby making the molecule.

22. The method of claim 21, further comprising:
(d) testing the molecule produced in step (c) for one or more of the following activities:
(i) removing copper from SOD1;
(ii) inhibiting catalytic activity of SOD1,
(iii) inhibiting proliferation of activated endothelial cells in vitro,
(iv) inhibiting proliferation of tumor cells in vitro;
(v) inhibiting endothelial cell migration in vitro or in vivo;
(vi) inhibiting tumor cell growth in vivo.

23. A method for removing copper from the SOD1 enzyme, comprising contacting a sample comprising SOD1 enzyme with an effective amount of a compound identified in accordance with the method of claim 1, for a time sufficient for removal of the copper from said enzyme.

24. The method of claim 23 wherein the compound is selected from the group consisting of ATN-427, ATN-714, ATN-719 and ATN-722.

25. The method of claim 23 wherein said removing is in vivo.

26. A method for inhibiting the activity of the SOD1 enzyme, comprising contacting a sample comprising SOD1 enzyme with an effective amount of a compound identified as an SOD1 inhibitor in accordance with the method of claim 1, for a time sufficient for inhibition of said enzyme.

27. The method of claim 26 wherein the compound is selected from the group consisting of ATN-427, ATN-714, ATN-719 and ATN-722.

28. The method of claim 26 wherein said contacting is in vivo.

29. A method for inhibiting endothelial cell proliferation comprising providing to endothelial cells an effective amount of a compound identified as a proliferation inhibitor in accordance with the method of claim 1, for a time sufficient for inhibition of said proliferation.

30. The method of claim 29 wherein the compound is selected from the group consisting of ATN-427, ATN-714, ATN-719 and ATN-722.

31. The method of claim 29 wherein said providing is in vivo.
